(19) United States
(12) Patent Application Publication
(10) Pub. No.: US 2021/0308063 A1
(43) Pub. Date: Oct. 7, 2021

(54) NOVEL ORAL COMPOSITION

(71) Applicant: APILLET APS, Roskilde (DK)

(72) Inventors: Carsten Lunde PETERSEN, Roskilde (DK); Anders CHRISTENSEN, Roskilde (DK)

(73) Assignee: APILLET APS, Roskilde (DK)

(21) Appl. No.: 17/262,386

(22) PCT Filed: Aug. 13, 2019
(86) PCT No.: PCT/EP2019/071667

§ 371 Date: Jan. 22, 2021

(30) Foreign Application Priority Data
Aug. 14, 2018 (EP) 18188848.8

(51) Int. Cl. A61K 9/48; A61K 9/00; A61K 45/06; A23L 33/00; A61K 9/28

(52) U.S. Cl. CPC A61K 9/4891; A61K 9/0053; A61K 9/2866; A23L 33/30; A61K 45/06

(57) ABSTRACT

A solid oral composition for targeted release in an intestine of a mammal comprising a core and a coating completely surrounding the core. The core includes an entrapped material to be released in the intestine, and the coating or part of the coating includes a first and a second component, and the first component is resistant to the environment in the stomach of a mammal and the second component enzymatically digest the first component when subjected to the more basic environment of the intestine compared to the more acidic environment of the stomach.

### TECHNICAL FIELD

The present invention relates to a solid oral composition for targeted release in an intestine of a mammal. Moreover, the present invention is concerned with the solid oral composition for different uses, such as for use as a medicinal product, for use as a nutritional supplement, or for use as a tracing agent. Furthermore, the present invention relates to the use of a pair of components for preparing a coating for a solid oral dosage form.

### BACKGROUND ART

Oral delivery of active pharmaceutical ingredients is often the chosen route of delivery since it is easier, more convenient and generally painless, resulting in larger patient adherence relative to other means of delivery.

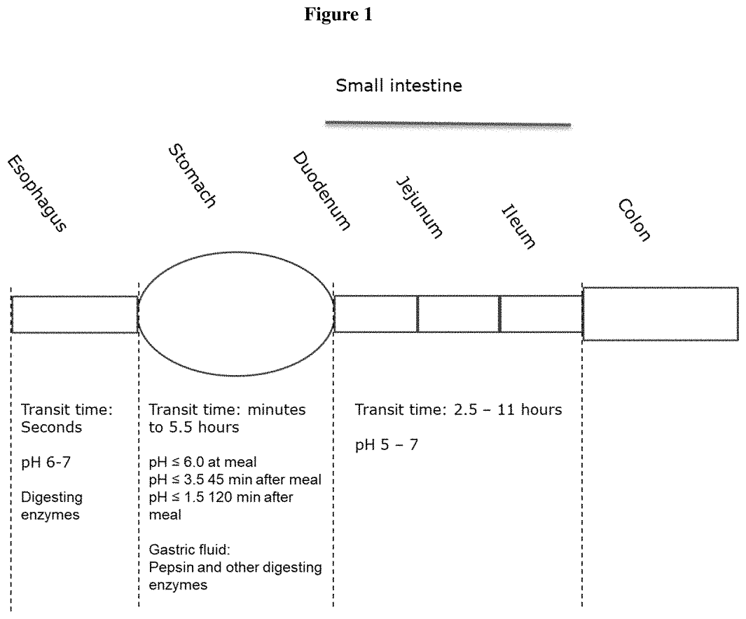

Therapeutically active and other important biological compounds may be administered to patients in a variety of ways, including for example, by the oral route. To administer proteins and peptides, but also sensitive small molecule therapeutics by the oral route is a challenge and has been pursued for many years. Some of the challenges include, but are not limited to, digestive enzymes and the strongly acidic environment of the stomach, components of pancreatic juices and secretions of the biliary system. Because of these challenges an unreliable bioavailability may be the consequence if sensitive active pharmaceutical ingredients are orally delivered. In addition, the compositions and concentrations of the gastro intestinal (GI) components are not uniform but vary within the segments of the GI tract. Some of the challenges for peptides, proteins and other sensitive substances in the GI tract are depicted in FIG. 1.

One skilled in the art will appreciate that, to increase efficiency and bioavailability, the dosage form should deliver the active agent (entrapped material), and any required excipient, to the part of the GI tract best suited chemically and physically for absorption of that active ingredient. There is a need for enteric coatings that can provide suitable bioavailability of such administered proteins, peptides and small molecules.

### SUMMARY OF THE INVENTION

In a first aspect the present invention relates to a solid oral composition for targeted release in an intestine of a mammal comprising a core and a coating completely surrounding the core, wherein the core comprises an entrapped material to be released in the intestine, and wherein the coating or part of the coating comprises a first and a second component, wherein the first component is resistant to the environment in the stomach of a mammal and the second component enzymatically digest the first component when subjected to the more basic environment of the intestine compared to the more acidic environment of the stomach.

In a second aspect the present invention relates to a solid oral composition for targeted release in an intestine of a mammal comprising a core and a coating completely surrounding the core, wherein the core comprises an entrapped material to be released in the intestine, and the coating or a part of the coating is adapted to be digested/to self-perforate in the intestine, wherein the coating comprises a first and a second component, and the first component is resistant to an environment in the stomach of a mammal and the second component enzymatically digest the first component when subjected to the more basic environment of the intestine compared to the more acidic environment of the stomach.

Typically, the coating substantially consists of a first and a second component.

In one embodiment the intestine is selected from small intestine, large intestine, duodenum, ileum, jejunum, and colon.

In a further embodiment the coating is adapted to resist breakdown from the environment in the stomach of a mammal.

In a still further embodiment the coating prevents the release of the entrapped material in the stomach of a mammal.

In a further embodiment the coating protects the entrapped material in the stomach of a mammal.

In a still further embodiment the coating is adapted to release the entrapped material in an intestine of a mammal.

As mentioned above the coating comprises a pair of components, in particular the pair of components are in contact with each other in the coating. When the coating consists of two components such components are in contact with each other, with no intermediate layer, to be ready to initiate the reaction in the intestine as explained in detail herein.

In a still further embodiment the first component is resistant to the environment in the stomach of a mammal and the second component enzymatically digest the first component when subjected to the more basic environment of the intestine compared to the more acidic environment of the stomach.

In a further embodiment the digesting activity of the second component is inhibited in the environment in the stomach of a mammal.

In a still further embodiment the mammal is selected from animals with little or no ability to digest dietary fibers like cellulose, such as a human, a monkey, a pig, a dog, a human ape, a rodent and a cat.

In a further embodiment the entrapped material is a protein, enzyme, polypeptide, oligopeptide, peptide, deoxyribonucleic acid (DNA), ribonucleic acid (RNA), a small organic molecule of less than 900 Da, or a pro-drug of any one of these materials, is a nutritional supplement, or a microbial culture, or a microbial additive, or a vaccine. Further embodiments of the entrapped material are selected from:
a) Proteins, Human growth hormone (hGH), Calcitonin, Insulin, GLP-1 analogues, GLP1,
b) Peptides, Octreotide,
c) Oral vaccines, Oral cholera vaccine, Mycoplasma hyopneumoniae oral vaccine, Live bacterial cells as attenuated vaccines,
d) Small organic molecules 200<MW<900 g/mol, Desmopressin, Vasopressin, Cyclosporine, Ranitidine, Diclofenac, Ketoprofen, Amifostine, Omeprazole, Gemcitabine, Domperidone, Paclitaxel, Cinnarizine, Donepezil, Leucovorin, Raloxifene, Indomethacin, Dextromethorphan, Nizatidine, Peptide Val-Leu-Pro-Val-pro-Arg (VLPVPR), Flurbiprofen, Mebendazole, Thymidine, Zolpidem tartarate, Loratidine, Venlafaxine, Tamsulosin, Urapidil, Prednisolone, Miconazole, Diltiazem, Ambroxol, Captopril, Acyclovir, Cimetidine, Metoprolol, Griseofulvin, Atazanavir, Ibuprofen, Azithromycin, Lercanidipine, Sulfacetamide, Azelastine, Zidovudine, Cloricromene, Oxymatrine, Acarbose, Propranolol, Alfuzosin, Stavudine, Lobenzarit, Genistein, Verapamil, Terbinafine, Lornoxicam, Clotrimazole.

In a still further embodiment the first component and the second component are mixed.

In a further embodiment the first component and the second component are in different layers with the first component being the outer layer and the second component the inner layer around the core.

In a still further embodiment the pair of the first and second component is selected from dietary fibers and enzymes that digest dietary fibers like cellulose and cellulase, pectin and pectinolytic enzymes, hemicellulose and hemicellulase, lignin and lignin degrading enzymes, fructan and fructan degrading enzymes, and lipid and lipases.

In a further embodiment the pair of the first and second component is selected from structurally ordered cellulose I, such as bacterial cellulose or derivatives thereof and a cellulase, such as cellulase (EC 3.2.1.4) (endocellulase) and cellubiase (EC 3.2.1.21) (beta-glucosidase) and 1,4-beta-cellobiosidase (EC 3.2.1.91) (Exocellulase). Further usable cellulases are Cellulose 1,4-beta-cellobiosidase (reducing end) (EC 3.2.1.176) (exocellulase) as well as cellulase complexes and mixtures thereof.

It is to be understood that the expression “cellulase” as used herein has the meaning understood by the person skilled in the art and covers all cellulases, such as endocellulase, exocellulase, cellulase complex, beta-glucosidase, and mixtures hereof.

In a still further embodiment, the first component is a cellulose and the second component is a cellulase selected from one or more of endocellulases, exocellulases, and beta-glucosidase. Preferably the first component comprises a structurally ordered cellulose I, such as a bacterial cellulose and the second component comprises an endocellulase. Typically, the cellulase, such as the endocellulase, has a cellulose binding domain and is active towards bacterial cellulose.

In a further embodiment the first component is a cellulose and the second component is a cellulase complex.

In a still further embodiment, the second component is enzymatically active and digests the first component in the environment in the intestine of a mammal. Typically, the second component has maximum enzymatic activity in the range pH 3-12, such as from pH 3.5 to 9.5, or from pH 3.0 to 9.0, or from pH 4.0 to 9.0.

In a further embodiment the composition is a tablet or a capsule or other type of solid oral dosage form.

In a third aspect the present invention relates to a composition of the first or second aspect or any one of the above embodiments, for use as a medicinal product. In one embodiment the medicinal product is in the form of a microbial culture.

In a fourth aspect the present invention relates to a composition of the first or second aspect or any one of the above embodiments for use as a nutritional supplement.

In a fifth aspect the present invention relates to a composition of the first or second aspect or any one of the above embodiments for use as a tracing agent.

In a further aspect the present invention relates to use of a pair of components for preparing a coating for a solid oral dosage form where the coating is degraded when subjected to a pH change from a lower to a higher pH, wherein the digestion of the first component by the second component is inhibited at the lower pH and the second component digest the first component when subjected to the higher pH.

### DESCRIPTION OF THE INVENTION

The present invention relates to a solid oral composition for targeted release in an intestine of a mammal. The solid oral composition may be any form known to the skilled person and in particular is a tablet or a capsule, to be administered via the oral route to a mammal. In one embodiment the oral composition is a tablet. In another embodiment the oral composition is a capsule. The mammal can be any mammal, such as a human.

The term “mammal” as used herein means animals, such as without limitation humans, dog, cat, pig, monkey, human apes, rodents. The term “mammal with little or no ability to digest cellulose” means human, dog, cat, pig, monkey, human apes, rodents and other animals with little or no ability to digest cellulose. Mammals, such as cows, cattle, horse, goat, sheep and deer, that natively have a digestion system with ability to digest cellulose polymers do not benefit from the present invention when the coating contains celluloses.

The solid oral composition for targeted release in the intestine of a mammal, such as a human, refers to the design of the solid composition as explained in detail below, which design allows accurate sensing of when the composition reaches the intestine, which is the target, hence targeted release.

The solid composition comprises a core, which core may contain a solution, suspension or be a solid core, provided the core itself does not have any influence on the stability of the coating. The core is surrounded by a coating which defines the outer solid layer, hence the overall composition is referred to as a solid composition. In the core a material is contained, and entrapped, which material is to be released in the intestine upon the coating or part of the coating being dissolved or destroyed. The entrapped material can be any material suitable for release in the intestine, such as without limitation, a drug, a nutrient, a supplement, a microbial organism, a vaccine, a radio transmitter, a device for measuring parameters in the intestine, etc. The coating or a part of the coating is adapted to be digested in the intestine, which means that the coating may consist of different coatings, such as a part to be digested and release the entrapped material and another part which remains intact, or the whole coating is to be digested or perforated in the intestine and release the entrapped material. The intestine comprises different elements, such as the small intestine, large intestine which consists of different elements duodenum, ileum, jejunum, and colon. It is intended that each of these elements can be subject to an embodiment in combination with the aspects and embodiments of the present invention.

Preferably, the coating is adapted to resist breakdown from the environment in a stomach of a mammal, so that no release of the entrapped material takes place in the stomach. The coating may in itself be composed of two or more elements where one enzymatically digests the other in the intestine, but not in the stomach. Thus, the coating prevents the release of the entrapped material in a stomach of a mammal, and/or the coating protects the entrapped material in a stomach of a mammal. When stated that the coating is adapted to release the entrapped material in an intestine of a mammal, it refers to the composition of the coating which composition will be disrupted by enzymatical digestion in the intestine. Such a coating is composed of two or more components, and in accordance with a preferred embodiment of the present invention the coating comprises a pair of components. The pair of components means two components and such components are different in that one must digest the other in order to make this a coating as used in accordance with the invention, wherein the coating or a part of the coating is adapted to be digested in the intestine.

The first component is resistant to the environment in the stomach of a mammal and the second component digest the first component when subjected to the more basic environment of the intestine compared to the more acidic environment of the stomach. In this respect the mammal is preferably selected from animals with little or no ability to digest cellulose, such as a human, a monkey, a pig, a dog, a human ape, a rodent and a cat. In a preferred embodiment the mammal is a human. The reference to first and second is only to indicate that the two components are different but does in no way refer to the way or any order of making the coating. Preferably, the second component digests the first component by having a digesting activity that is inhibited in the environment in the stomach of the mammal. The second component if not inhibited in the environment in the stomach may become a less workable solution although this could be compensated for by adding a further coating covering the solid oral composition of the present invention.

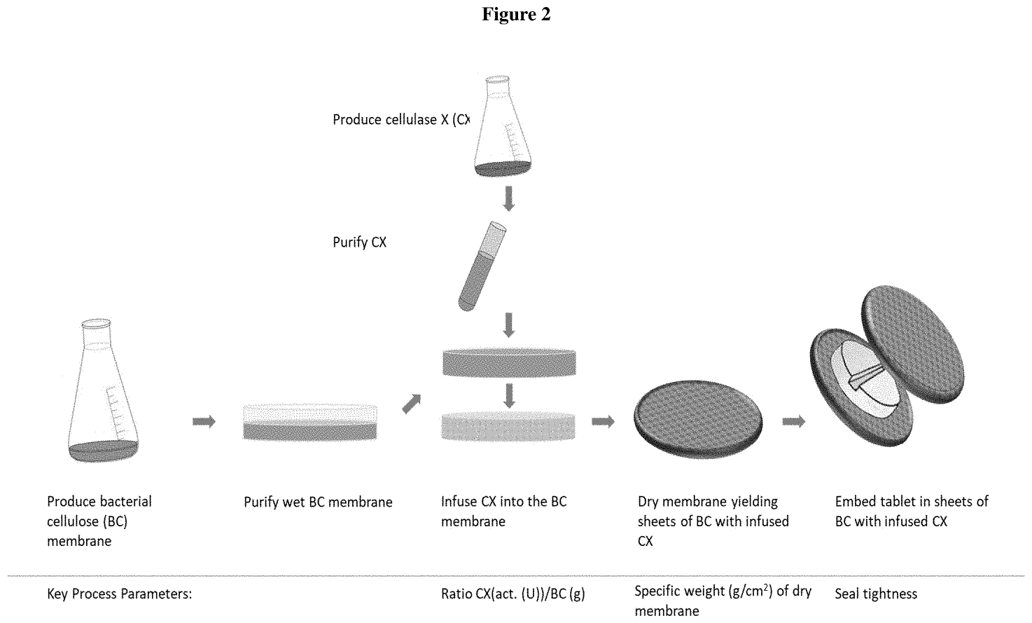

One method of making a coating for use in accordance with the present invention is the preparation of the pair of components. The first component of the coating composition for use in the present invention can for instance be produced by first producing a bacterial cellulose (BC) cuticle by microbial fermentation, secondly purify the BC. This procedure may include an alkaline treatment step. Then the second component, such as a cellulase can be produced by microbial fermentation, chemical synthesis or other means, followed by a purification of this cellulase, for instance by purifying by affinity chromatography. Hereafter, the cellulase is made in a fluid formulation and infused into the BC cuticle (for instance at a pH or at other conditions where the cellulase is inactive). Dry the cuticle to obtain sheets of BC impermeable to molecules of MW>200 Da containing the cellulase (CX). Embed core (comprising the entrapped material to be released in the intestine) in the BC/CX sheets and seal with an appropriate component. An outline of a coating production process is shown in FIG. 2.

The entrapped material can be anything which is suitable for release in the intestine, such as compounds for use as medicine.

In a further embodiment the entrapped material is a drug. In a still further embodiment the entrapped material is a nutritional supplement. In a further embodiment the entrapped material is a microbial culture. In a still further embodiment the entrapped material is a microbial additive. In a further embodiment the entrapped material is a vaccine.

Further embodiments of the drug are selected from one or more of a protein, an enzyme, a polypeptide, an oligopeptide, a peptide, a deoxyribonucleic acid (DNA), ribonucleic acid (RNA), a small organic molecule of less than 900 Da, or a pro-drug of any one of these materials, and all of these are considered individual embodiments and may be subject to one or more claims in respect of any one of the aspects and embodiments of the aspects described herein.

Further embodiments of the entrapped material are selected from one or more of:
a) Proteins, Human growth hormone (hGH), Calcitonin, Insulin, GLP-1 analogues, GLP-1,
b) Peptides, Octreotide,
c) Oral vaccines, oral cholera vaccine, Mycoplasma hyopneumoniae oral vaccine, live bacterial cells as attenuated vaccines,
d) Small organic molecules 200<MW<900 g/mol, Desmopressin, Vasopressin, Cyclo-sporine, Ranitidine, Diclofenac, Ketoprofen, Amifostine, Omeprazole, Gemcitabine, Domperidone, Paclitaxel, Cinnarizine, Donepezil, Leucovorin, Raloxifene, Indomethacin, Dextromethorphan, Nizatidine, Peptide Val-Leu-Pro-Val-pro-Arg (VLPVPR), Flurbiprofen, Mebendazole, Thymidine, Zolpidem tartarate, Loratidine, Venlafaxine, Tamsulosin, Urapidil, Prednisolone, Miconazole, Diltiazem, Ambroxol, Captopril, Acyclovir, Cimetidine, Metoprolol, Griseofulvin, Atazanavir, Ibuprofen, Azithromycin, Lercanidipine, Sulfacetamide, Azelastine, Zidovudine, Cloricromene, Oxymatrine, Acarbose, Propranolol, Alfuzosin, Stavudine, Loben-zarit, Genistein, Verapamil, Terbinafine, Lornoxicam, Clotrimazole. Each of the compounds or vaccines are considered individual embodiments and may be subject to one or more claims in respect of any one of the aspects and embodiments of the aspects described herein.

The pair of components may be mixed or may be in separate layers. Other ways of making a coating consisting of two components are contemplated by the invention as long as the two components are in contact with each other. For instance, the first component and the second component are mixed. Alternatively, the first component and the second component are in different layers, such as two layers wherein the first component being the outer layer and the second component the inner layer around the core.

In a still further embodiment the pair of the first and second component is dietary fibers and enzymes that digest dietary fibers. In a further embodiment the pair of the first and second component is cellulose and cellulase. In a further embodiment the pair of the first and second component is pectin and pectinolytic enzymes. In a still further embodiment the pair of the first and second component is hemicellulose and hemicellulase. In a further embodiment, the pair of the first and second component is lignin and lignin degrading enzymes. In a still further embodiment the pair of the first and second component is fructan and fructan degrading enzymes. In a further embodiment the pair of the first and second component is lipid and lipases.

When the pair of the first and second component is cellulose and cellulase, preferably, the first and second component is selected from structurally ordered cellulose I and a cellulase. The most abundant type of native cellulose found in nature is called cellulose I [6, 7, 8]. The structurally ordered cellulose I is preferably, a bacterial cellulose or derivatives thereof and the cellulase is preferably selected from the group consisting of cellulase (EC 3.2.1.4) (endocellulase), cellubiase (EC 3.2.1.21) (beta-glucosidase) 1,4-beta-cellobiosidase (EC 3.2.1.91) (Exocellulase), Cellulose 1,4-beta-cellobiosidase (reducing end) (EC 3.2.1.176) (exocellulase) as well as cellulase complexes and mixtures thereof.

The term “structurally ordered cellulose I” or “native cellulose” as used interchangeable herein refers to an unbranched polymer containing any number of eight or more D-glucose units, linked by β-1,4 glycosidic bonds.

In a still further embodiment the second component is enzymatically active and di-gests the first component in the environment in the intestine of a mammal. In particular, the second component has maximum enzymatic activity in the range pH 3-12, such as pH 3.5 to 9.5, or 3.0 to 9.0, or 4.0 to 9.0.

Furthermore, the present invention relates to a solid oral composition for targeted release in an intestine of a mammal comprising a core and a coating completely surrounding the core, wherein the core comprises an entrapped material to be released in the intestine, and the coating or a part of the coating is adapted to be digested in the intestine, for use as a medicinal product. Each of the above described embodiments in relation to the first aspect also applies to this particular use aspect.

Furthermore, the present invention relates to a solid oral composition for targeted release in an intestine of a mammal comprising a core and a coating completely surrounding the core, wherein the core comprises an entrapped material to be released in the intestine, and the coating or a part of the coating is adapted to be digested in the intestine, wherein the coating comprises a first and a second component, and the first component is resistant to an environment in the stomach of a mammal and the second component enzymatically digest the first component when subjected to the more basic environment of the intestine compared to the more acidic environment of the stomach, for use as a nutritional supplement. Each of the above described embodiments in relation to the first aspect also applies to this particular use aspect.

Furthermore, the present invention relates to a solid oral composition for targeted release in an intestine of a mammal comprising a core and a coating completely surrounding the core, wherein the core comprises an entrapped material to be released in the intestine, and the coating or a part of the coating is adapted to be digested in the intestine, wherein the coating comprises a first and a second component, and the first component is resistant to an environment in the stomach of a mammal and the second component enzymatically digest the first component when subjected to the more basic environment of the intestine compared to the more acidic environment of the stomach, for use as a tracing agent. Each of the above described embodiments in relation to the first aspect also applies to this particular use aspect.

In a further aspect the present invention relates to use of a pair of components for preparing a coating for a solid oral dosage form where the coating is degraded when subjected to a pH change from a lower to a higher pH, wherein the first component is adapted to resist digestion at the lower pH and the second component digest the first component when subjected to the higher pH. The coating may be used to surround the solid oral dosage form or cover a part of the dosage form as the case may be. Typically, the solid oral dosage form is a tablet, a capsule or granulates. Typically, the lower pH is in the range 1.0-4.5 and the higher pH is in the range 4.5-8.5 in the intestine. The creation of the coating according to this further aspect which coating can be stored for later use is also contemplated by this aspect of the invention. The coating can be used in setting up test systems to verify a suitable pair of components and amounts and ratios to be used.

In a further aspect, the present invention relates to a composition comprising a pair of components, wherein a) the first component is resistant to an environment i) resembling the stomach of a mammal or ii) in the stomach of a mammal and b) the second component enzymatically digest the first component when subjected to i) the more basic environment of the intestine or ii) to a basic environment resembling the environment in the intestine compared to the more acidic environment of the stomach or more acidic environment resembling the stomach.

The above embodiments should be seen as referring to any one of the aspects (such as ‘composition, ‘pharmaceutical composition’, ‘composition for use as a medicinal product, or ‘compound for use in a method’) described herein as well as any one of the embodiments described herein unless it is specified that an embodiment relates to a certain aspect or aspects of the present invention.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

The term “and/or” as used herein is intended to mean both alternatives as well as each of the alternatives individually. For instance, expression “xxx and/or yyy” means “the xxx and yyy; the xxx; or the yyy”, all three alternatives are subject to individual embodiments.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

The present invention is further illustrated by the following examples that, however, are not to be construed as limiting the scope of protection. The features disclosed in the foregoing description and in the following examples may, both separately and in any combination thereof, be material for realizing the invention in diverse forms thereof.

Experimental Procedures

Abbreviations

| | |
|---|---|
| BC | Bacterial cellulose |
| CCH | Cumulated cellulose hydrolysis |
| mma | Minutes of maximal activity |
| % ma | Percentage of maximal activity |
| pHd | pH duodenum |
| pHs | pH stomach |
| SRB | Sulforrodamin B |
| PBS | Phosphate buffered saline |
| HCl | Hydrochloride |
| OD | Optical density |
| h | Hours |
| nm | Nanometer |
| DNS | Dinitrosalicylic |
| MW | Molecular weight |
| St. Dev. | Standard deviation |

Methods and Results

Physiological parameters were extracted for gastro intestinal pH and passage times in humans from literature. From these parameters, a semi-physiological model of the pH in the GI tract was synthesized and validated against literature data. Further, we extracted biochemical parameters for cellulases characterized by having activity maxima around neutral pH and strongly inhibited activity at acidic pH from the Brenda bioinformatics database [9] and from literature [10, 11, 12]. From these data a semi-biochemical model of the pH dependency of cellulase activity was synthesized and validated against literature data. By combining these two models a semi-physiological model of cumulated cellulase activity was synthesized and used to perform simulations of cellulase passing through the human GI tract.

Results: The two synthesized models predicted the literature data well. Simulations showed that the cellulase activity is strongly inhibited as the cellulase passes through the stomach and that the cellulase becomes active when entering the small intestine. Further, simulations showed only a small amount of cumulated activity (cumulated cellulose breakdown) takes place during the passage of the stomach compared to the cumulated activity (cumulated cellulose breakdown) that takes place during the passage of the small intestine, even at physiologically highly prolonged stomach passage times and physiologically very short small intestine passage time. This shows there is a robust design space for a cellulose-cellulase based coating which protects the entrapped material during the passage of the stomach and which perforates in the intestine.

The entero coating presented here is a method which robustly protects an entrapped material during the passage of the stomach and which robustly releases the material in the intestine through a self-perforating mechanism.

Results

Cellulases with a maximal activity in the pH range 5.5-8.0 and with activity at most 25% of maximal activity (% ma) in acidic environments (pH<4.0) were identified in the Brenda bioinformatics database and in literature [9, 10, 11, 12] and biochemical characterization data from these cellulases were used to synthesize a model (A₁ (pH)) predicting the relative cellulase activity as a function of pH.

$${A}_{1} ( pH ) = 100 * exp ( - {( \frac{\vert pH - 6.5 \vert}{2} )}^{3} ) \% ma$$

The synthesised activity function A₁ (pH) models a cellulase with maximal activity at pH 6.5.

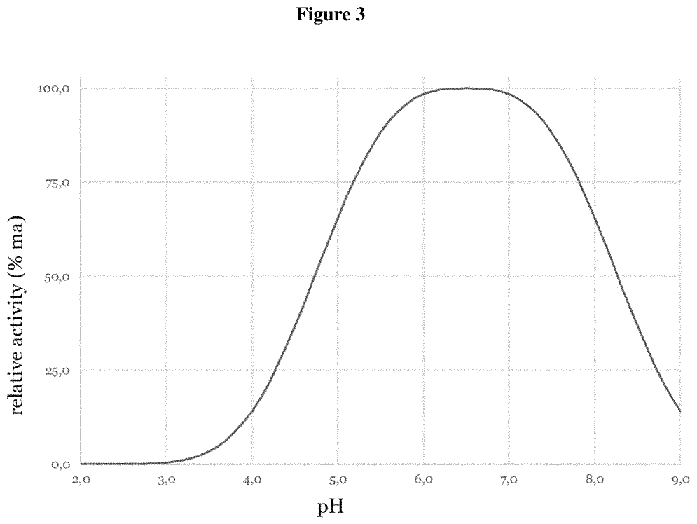

A graphic representation of the relative activity as a function of pH is shown in FIG. 3. The model was validated against literature data by overlaying the synthesized A₁ (pH) activity function with the activity data from cellulases identified in the literature [10, 11, 12].

A model for pH(t) in the human gastro intestinal system was synthesized by combining physiological data from the literature [1, 2].

The model for the variation of pH in the stomach (pHs(t)) of healthy young men and women with time after meal ingression was extracted from the study [1].

pH_{S}(t)=0.5+exp(−0.016*t+1.7)

The model with constant pH=6.1 for the pH in the small intestine is based on the study [2]. Denoting by T_{S} the stomach transit time and by T₁ the intestinal transit time we obtain a model pH(t) predicting the human gastro intestinal pH environment surrounding a capsule/an entrapped material as it passes through the intestine. FIG. 1 shows a graphic representation of the synthesized model A₁ (pH) predicting the relative cellulase activity as a function of pH.

$$pH ( t ) = \{ \begin{matrix}0.5 + exp ( - 0.016 * t + 1 .7 ) , & 0 \leq t \leq {T}_{S} , \\ 6.1 , & {T}_{S} \leq t \leq {T}_{S} + {T}_{I}\end{matrix} .$$

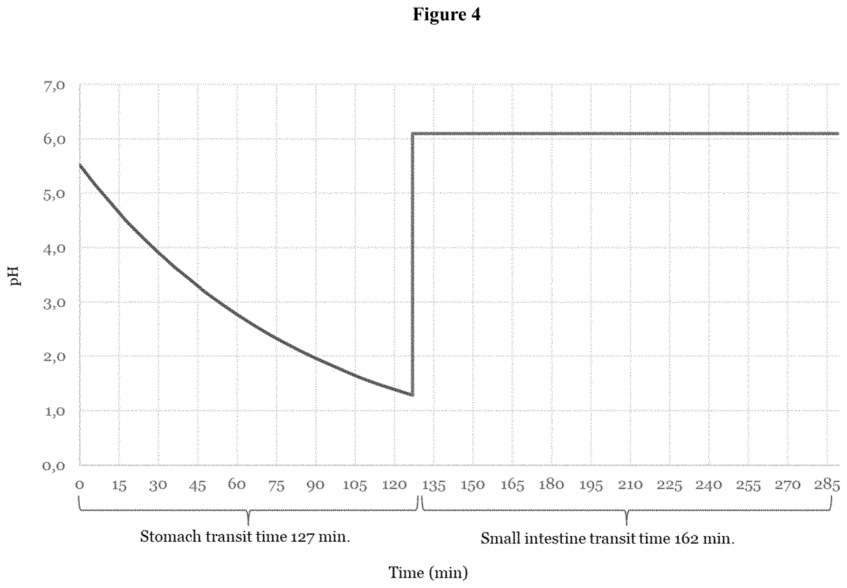

The model pH(t) was plotted on top of human stomach and intestinal physiological data and predicts well the data on gastro intestinal pH in healthy humans found in the literature [1, 2]. FIG. 4 shows the graphic representation of the model predicting the gastro intestinal pH(t) in humans with a stomach transition time T_{S} of 127 min and a short small intestine transit time T₁ of 162 min.

By combining the model (A₁ (pH)) predicting the relative cellulase activity as a function of pH with the model predicting the gastro intestinal pH(t) in humans, a model of relative cellulase activity in the GI tract was obtained by composing the two functions:

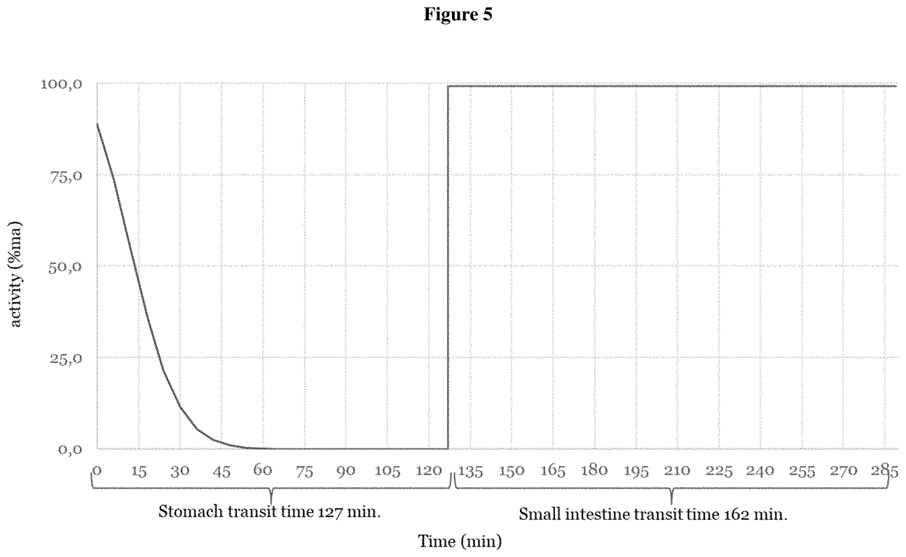

A₁(pH(t))

Where A₁ (pH) is the cellulase activity function and pH(t) is the human gastro intestinal pH function. FIG. 5 shows a graphic representation of the model of relative cellulase activity in the gastro intestinal tract. The relative cellulase activity in the GI tract with a stomach transition time T_{S} of 127 min and a small intestine transit time T₁ of 162 min is shown.

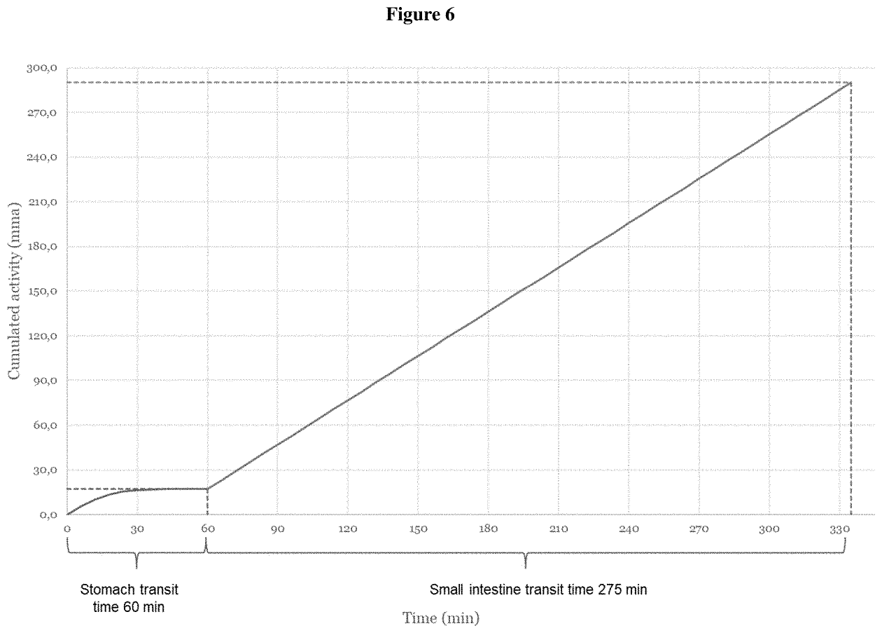

A model predicting the cumulated activity in the self-perforating material, where the first component is cellulose, and the second component is a cellulase, is obtained by integrating the relative activity function A₁(pH(t))/100 with respect to time. A graphic representation of the cumulated activity in the self-perforating material with a median stomach transition time T_{S} of 60 min and a median small intestine transit time T₁ of 275 min is shown in FIG. 6.

For the calculations of cumulated activity, a relative unit minutes of maximal activity (mma) is used. The mma unit describes the cumulated cellulase activity as equivalents of reaction minutes when the reaction is performed at optimal conditions. e.g. at pH 6.5, the optimal reaction pH, in the case of the model cellulase described in A₁ (pH).
The basic unit 1 mma is the enzymatic cellulase turnover of β-1,4-glycosidic bonds at maximal enzymatic activity for one minute.
The mma unit is related to a specific number of active sites e.g. amount of cellulase and a specific number of 1-4 glycosidic bounds, e.g. amount of cellulose
The mma unit is specific to individual cellulases and to individual cellulose substrates

The mma unit can be used to compare the cumulated cellulase activity under different reaction conditions, for example reaction time, temperature, pH, cofactor concentration and inhibitor concentrations. The mma unit is convenient for product design and may be used as a production specification to design a material which self-perforate within a certain range of cumulated cellulase activity.

### Example 1

The model of the performance of the self-perforating material in the gastro-intestinal system A₁(pH(t))/100 was used to study the activity cumulating in the self-perforating material when passaging the human GI tract under different physiological conditions. Five studies were made to explore, under different stomach emptying times and small intestine transition times, the properties of the self-perforating material, where the first component is cellulose, and the second component is a cellulase with the activity function A₁ (pH).

### A Stomach Transition Time of 60 Min and a Small Intestine Transit Time of 275 Min

A typical median stomach emptying time was found in the literature to be 60 min [3,4,5] and a typical median intestinal transition time was found to be 275 min in the literature [3,4,5]. A graphic representation of the cumulated cellulose hydrolysis predicted by the model using these stomach-emptying and small intestine transition times is shown in FIG. 6. FIG. 2 shows the model predicting the cumulated activity in the self-perforating material in the gastrointestinal system. Input parameters: a stomach transition time T_{S} of 60 min and a small intestine transit time T₁ of 275 min.

The model of the cumulated activity in the GI tract shows that a self-perforating material designed to withstand a cumulated activity of more than 17 mma will protect the entrapped material from the environment in the stomach with a stomach emptying time of up to 60 min. In addition, a self-perforating material designed to be digested with a cumulated activity of less than 290 mma will release the entrapped material in the human small intestine given a small intestine transit time of 275 min. Thus, a self-perforating material designed to become digested by a cumulated activity in the range 17 to 290 mma will release the entrapped material with typical median stomach and small intestinal transit times.

### A Stomach Transition Time of 5 Min and a Small Intestine Transit Time of 162 Min

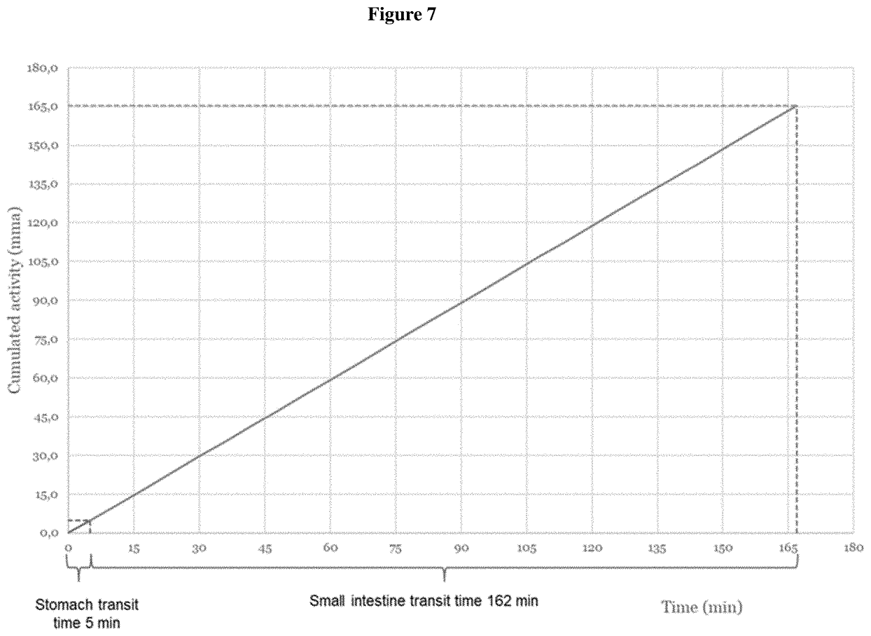

A typical short stomach emptying time was found in the literature to be 5 min [3,4,5] and a typical short intestinal transition time was found to be 162 min in the literature [3,4,5]. A graphic representation of the cumulated cellulose hydrolysis predicted by the model using these stomach-emptying and small intestine transition times is shown in FIG. 7. FIG. 3 shows the model predicting cumulated activity in the self-perforating material in the gastrointestinal system. Input parameters: a stomach transition time T_{S} of 5 min and a small intestine transit time T₁ of 162 min.

The model of the cumulated activity in the GI tract shows that a self-perforating material designed to withstand a cumulated activity of more than 5 mma will protect the entrapped material from the environment in the human stomach with a stomach emptying time of up to 5 min. In addition, when designed to be digested with a cumulated activity of less than 165 mma it will release the entrapped material in the human small intestine given a small intestine transit time of 162 min. Thus, a self-perforating material designed to become digested by a cumulated activity in the range 5 to 165 mma will release the entrapped material with typical short stomach and small intestinal transit times.

### A Stomach Transition Time of 337 Min and a Small Intestine Transit Time of 162 Min

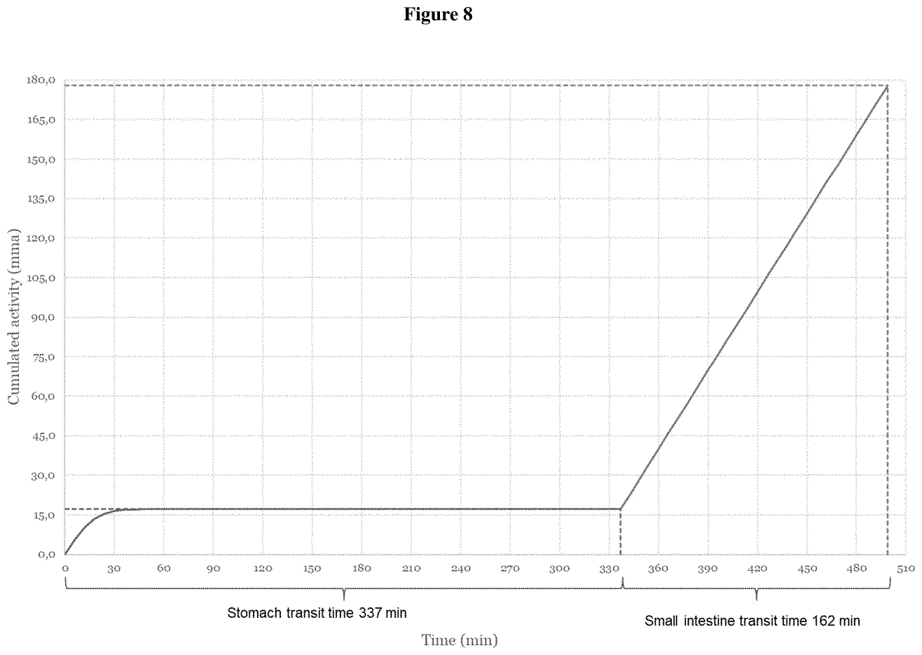

A typical long stomach emptying time was found in the literature to be 337 min [3,4,5] and a typical short intestinal transition time was found to be 162 min in the literature [3,4,5]. A graphic representation of the cumulated cellulose hydrolysis predicted by the model using these stomach-emptying and small intestine transition times is shown in FIG. 8. FIG. 8 shows the model predicting the cumulated activity in the self-perforating material in the gastrointestinal system using input parameters: stomach transition time T_{S} of 337 min; small intestine transit time T₁ of 162 min.

The model of the cumulated activity in the GI tract shows that a self-perforating material designed to withstand a cumulated activity of more than 17 mma will protect the entrapped material from the environment in the human stomach with a stomach emptying time of up to 337 min. In addition, if designed to be digested with a cumulated activity of less than 178 mma the entrapped material will be released in the human small intestine given a small intestine transit time of 162 min. Thus, a self-perforating material designed to become digested by a cumulated activity in the range 17 to 178 mma will release the entrapped material with typical long stomach and short small intestinal transit times.

### A Stomach Transition Time of 5 Min and a Small Intestine Transit Time of 669 Min

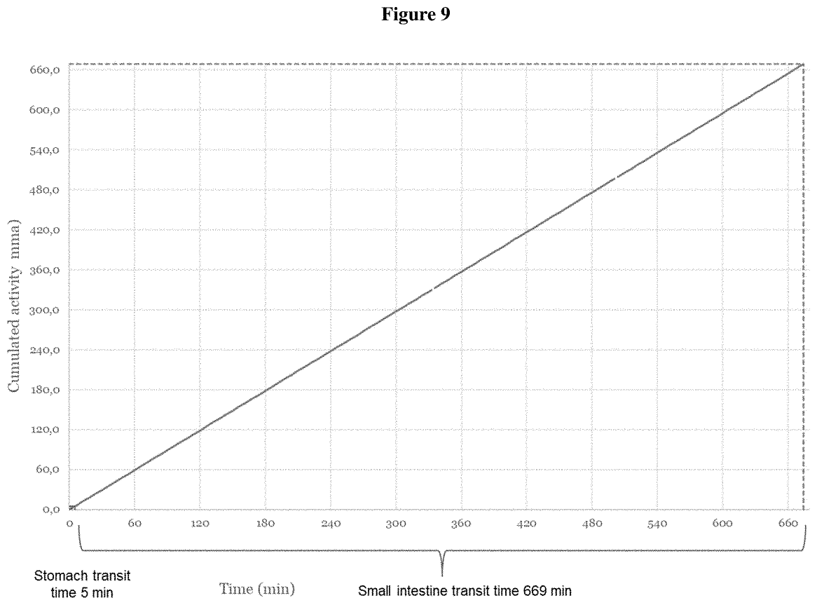

A typical short stomach emptying time was found in the literature to be 5 min [3,4,5] and a typical long intestinal transition time was found to be 669 min in the literature [3,4,5]. A graphic representation of the cumulated cellulose hydrolysis predicted by the model using these stomach-emptying and small intestine transition times is shown in FIG. 9. FIG. 9 shows the model predicting the cumulated activity in the self-perforating material in the gastro-intestinal system. Input parameters: a stomach transition time T_{S} of 5 min and a small intestine transit time T₁ of 669 min.

The model of the cumulated activity in the GI tract shows that a self-perforating material designed to withstand a cumulated activity of more than 5 mma will protect the entrapped material from the environment in the human stomach with a stomach emptying time of up to 5 min. In addition, a self-perforating material designed to be digested with a cumulated activity of less than 668 mma will release the entrapped material in the human small intestine given a small intestine transit time of 669 min Thus, a self-perforating material designed to become digested by a cumulated activity in the range 5 to 668 mma will release the entrapped material with typical long stomach and short small intestinal transit times.

### A Stomach Transition Time of 337 Min and a Small Intestine Transit Time of 669 Min

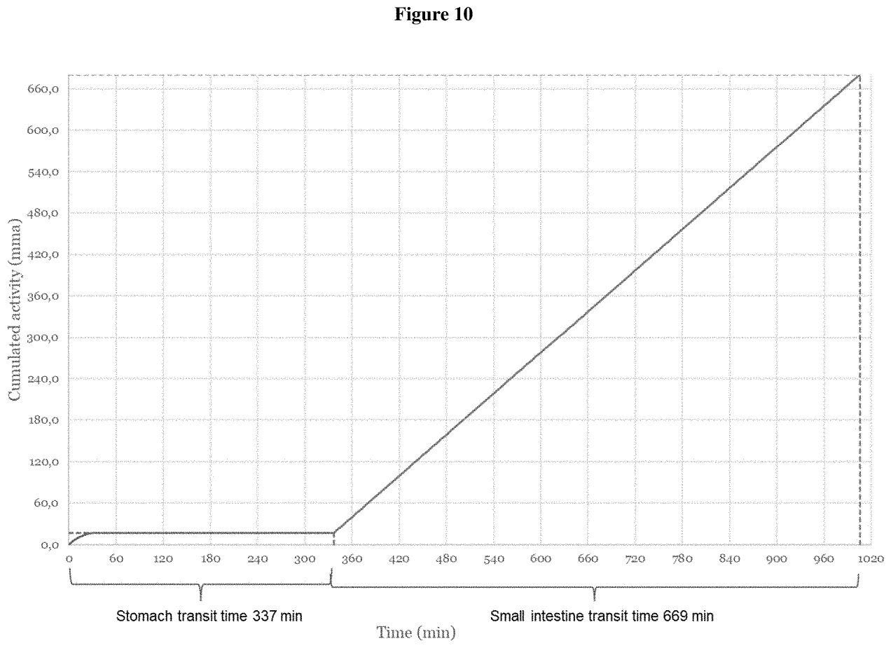

A typical long stomach emptying time was found in the literature to be 337 min [3,4,5] and a typical long intestinal transition time was found to be 669 min in the literature [3,4,5]. A graphic representation of the cumulated cellulose hydrolysis predicted by the model using these stomach-emptying and small intestine transition times is shown in FIG. 10. FIG. 10 shows the model predicting the cumulated activity in the self-perforating material in the gastro-intestinal system. Input parameters: a stomach transition time T_{S} of 337 min and a small intestine transit time T₁ of 669 min.

The model of the cumulated activity in the GI tract shows that a self-perforating material designed to withstand a cumulated activity of more than 17 mma will protect the entrapped material from the environment in the human stomach with a stomach emptying time of up to 337 min. In addition, a self-perforating material designed to be digested with a cumulated activity of less than 681 mma will release the entrapped material in the human small intestine given a small intestine transit time of 669 min. Thus, a self-perforating material designed to become digested by a cumulated activity in the range 17 to 681 mma will release the entrapped material with typical long stomach and long small intestinal transit times.

In the five studies in example 1 all combinations of typical short and long stomach emptying times and typical short and long intestinal transition times were studied. The self-perforating material, where the first component is cellulose, and the second component is a cellulase with the activity function A₁ (pH), can be designed to protect the entrapped material from the environment in the human stomach, and release the entrapped material in the human small intestine in any of the above considered cases if the material is designed with a cumulated activity specification in the window 17 mma to 165 mma.

### Example 2

The model predicting the performance of the self-perforating material in the gastrointestinal system A₁(pH(t))/100 was modified to study the performance of the self-perforating material in the human GI tract when the coating is given a time T_{PM} post meal ingression. The formula for the pH variation then becomes:

$$pH ( t ) = \{ \begin{matrix}0.5 + exp ( - 0.016 * ( {T}_{P M} + t ) + 1 .7 ) , & 0 \leq t \leq {T}_{S} , \\ 6.1 , & {T}_{S} \leq t \leq {T}_{S} + {T}_{I}\end{matrix} .$$

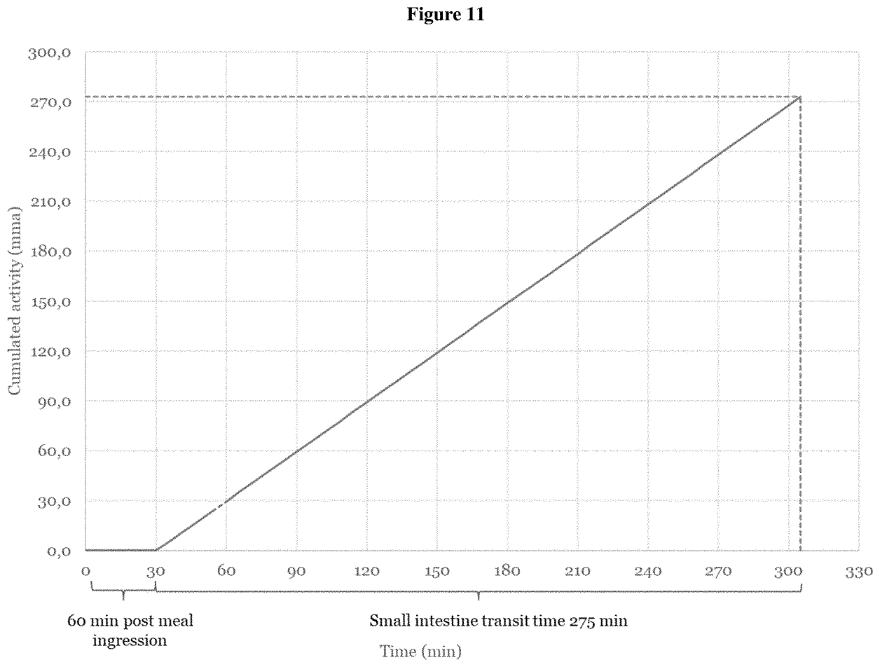

The obtained model was used to study the cumulated activity when the coating is given at T_{PM}=60 min post meal ingression and considering a stomach transit time T_{S} of 30 min. A graphic representation is shown in FIG. 11. FIG. 4 shows the cumulated activity in the self-perforating material in the human gastro intestinal tract when the material is given at T_{PM}=60 min post meal ingression and considering a stomach transit time T_{S} of 30 min and a small intestine transit time T_{S} of 275 min.

When the coating is given 60 min post meal ingression the cellulase activity is very low because of the acidic environment. This is reflected in the predicted cumulated activity of 0 mma in the stomach. When the coating material enters the small intestine the cellulase activity increases, initiating the breakdown of the coating, and supporting the release of the entrapped material. By ingression of the coating at a delayed time following a meal, the cumulated activity in the stomach is decreased significantly compared to when the coating is ingressed together with a meal. Thus, the design space for the coating is increased significantly.

### Example 3

The effects on the cumulated activity in the gastro intestinal tract of using cellulase enzymes with other pH optima was studied.

The model predicting the performance of the self-perforating material in the gastrointestinal system was modified to A₂ (pH(t))/100 to predict the performance in the human GI tract of a cellulose-cellulase self-perforating material, when the cellulase has maximal activity at pH_{OPT}. A₂ (pH) models a cellulase enzyme with characteristics similar to the cellulase properties described in model A₁ (pH), however, with an enzymatic optimum at pH_{OPT}.

$${A}_{2} ( pH ) = 100 * exp ( - {( \frac{\vert pH - p {H}_{O P T} \vert}{2} )}^{3} ) \% ma$$

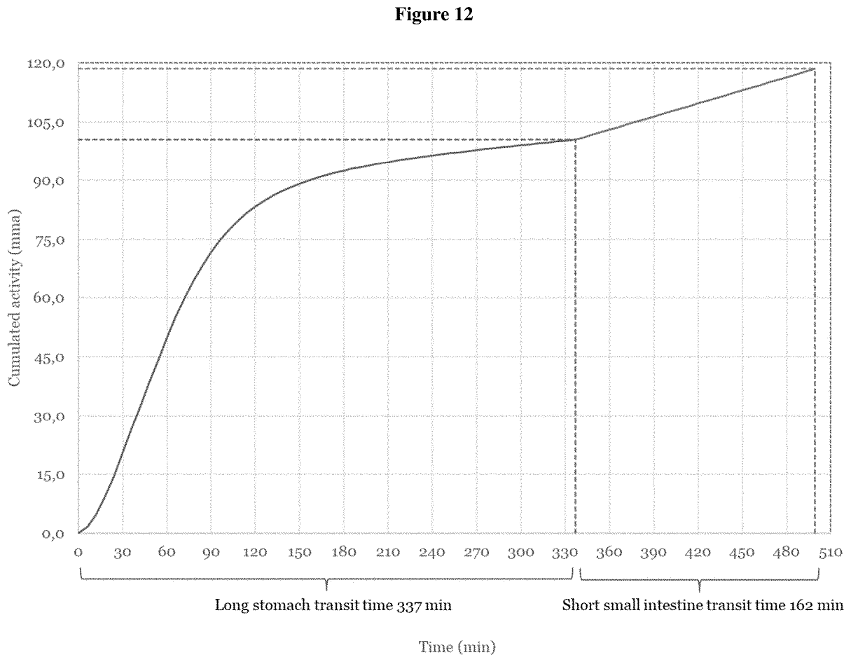

A₂ (pH) was used to study the cumulated activity of an enzyme with maximal activity at pH_{OPT} equal to 3.5. A graphic representation is shown in FIG. 12. FIG. 5 shows the graphic representation of the cumulated activity of an enzyme with maximal activity at pH_{OPT}=3.5 described in function A₂ (pH(t))/100.

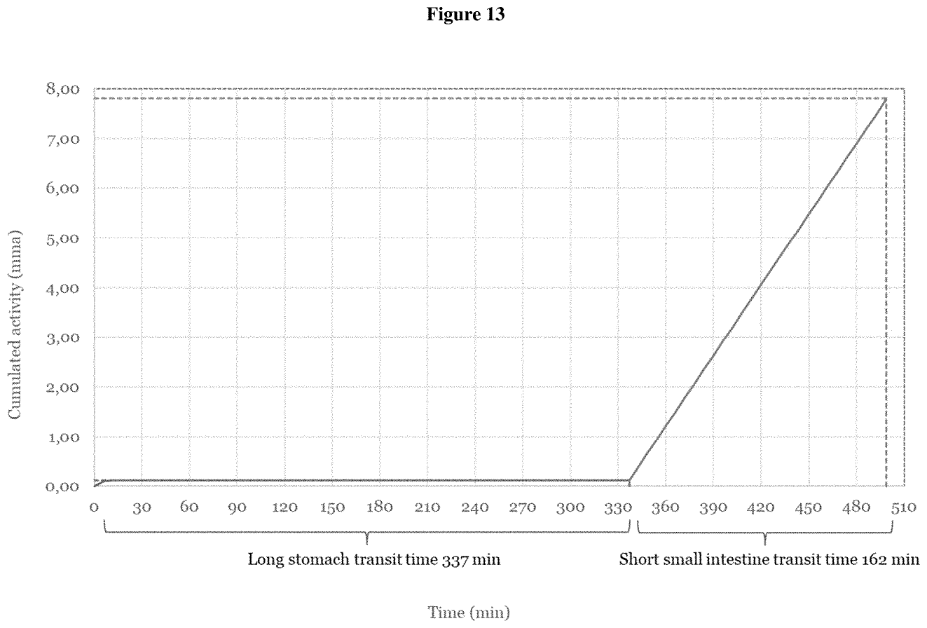

A₂ (pH) was used to study the cumulated activity of an enzyme with the maximal activity at pH_{OPT} equal to 9.0. A graphic representation is shown in FIG. 13. FIG. 6 shows the graphic representation of the cumulated activity of an enzyme with maximal activity at pH_{OPT}=9.0 described by function A₂ (pH(t))/100.

In a composition with a cellulase with maximal activity at pH 3.5 the cumulated activity in the stomach levels out at almost zero at prolonged stomach transit times (FIG. 12). A composition with an enzyme with maximal activity at pH 3.5 can protect the entrapped material in the stomach when the coating material is designed to release the entrapped material in the range 100 mma to 118 mma. However, such a composition with an enzyme with maximal activity at pH 3.5 may require that the coating is given post meal ingression to obtain a robust design space for release of the entrapped material in the small intestine.

A composition with an enzyme with maximal activity at pH 9.0 can protect the entrapped material in the stomach when the coating material is designed to release the entrapped material in the range 0.1 mma to 7.8 mma. A very robust design space for releasing the entrapped material in the intestine can be obtained with such a composition.

A composition with an enzyme with maximal activity above pH 9.0 will only improve the ability of the material to protect the entrapped material in the stomach and release the entrapped material in the intestine.

A composition with a cellulase enzyme with an enzymatic optimum in the range from at least pH 3.0 to 12.0, such as from 3.5 to 9.0, can serve as the second component in the self-perforating material, and support the protection of the entrapped material in the stomach and a release in the small intestine.

### Example 4

The purpose of this experiment was to determine the effect of the thickness (measured as specific mass mg/cm²) of a BC membrane and pH on the diffusion of a model compound through the membrane.

Wet BC membrane from cultures of Komagataebacter xylinus were compressed to remove water and then freeze-dried (3 days, −99° C.; 0.049 mbar). Membranes with a mass per area of 3.3 mg/cm² and 13.9 mg/cm² (thickness) were obtained. Sulforrodamin B (SRB) (MW 558.7 g/mol) was used as model compound. The diffusion over time of SRB through the BC membranes were investigated in a diffusion cell at pH 2 and pH 6.5. A diffusion cell consisting of a donor chamber where the model compound is placed at the beginning of the study, and a receptor chamber into which the model compound may migrate. The membrane to be studied is placed between the two chambers.

The donor chamber was filled with a 2 mg/mL SRB solution in 0.01 M HCl (pH 2) or a PBS (pH 6.5). The membrane to be studied was placed on top of the donor chamber. The study was initiated when the receptor chamber was filled with 0.01 M HCl (pH 2) or PBS (pH 6.5) as corresponding to the pH in the donor chamber. Samples were taken from the receptor chamber after 0 h, 1 h, 1.5 h, 2 h, 4 h and 6 h and the concentration of SRB was measured at OD 565 nm. The results are shown in table 1 and table 2.

TABLE 1
The concentration of the SRB model compound in the receptor
chamber (μg/ml) in a time cause experiment at pH 2.

| | SRB (μg/ml) in the receptor chamber at pH 2 | | | |
|---|---|---|---|---|
| | | 3.3 mg/cm² | | 13.9 mg/cm² |
| Diffusion | 3.3 mg/cm² | membrane | 13.9 mg/cm² | membrane St. |
| time (h) | membrane | St. Dev. | membrane | Dev. |
| 0.0 | 0.2 | 0.0 | 0.0 | 0.1 |
| 1.0 | 29.6 | 8.1 | 2.0 | 1.6 |
| 1.5 | 46.3 | 12.9 | 2.9 | 2.4 |
| 2.0 | 66.8 | 17.3 | 4.6 | 3.7 |
| 4.0 | 155.6 | 28.8 | 12.4 | 8.7 |
| 6.0 | 252.5 | 56.7 | 21.5 | 11.9 |

TABLE 2
The concentration of the SRB model compound in the receptor
chamber (μg/ml) in a time cause experiment at pH 6.5.

| | SRB (μg/ml) in the receptor chamber at pH 6.5 | | | |
|---|---|---|---|---|
| | | 3.3 mg/cm² | | 13.9 mg/cm² |
| Diffusion | 3.3 mg/cm² | membrane | 13.9 mg/cm² | membrane St. |
| time (h) | membrane | St. Dev. | membrane | Dev. |
| 0.0 | 0.1 | 0.1 | 0.2 | 0.2 |
| 1.0 | 19.8 | 8.5 | 2.5 | 1.2 |
| 1.5 | 31.3 | 13.5 | 4.9 | 2.5 |
| 2.0 | 46.3 | 16.5 | 7.9 | 4.5 |
| 4.0 | 97.5 | 15.1 | 23.3 | 11.6 |
| 6.0 | 142.3 | 16.2 | 39.2 | 16.7 |

The results in table 1 show that the SRB concentration in the receptor chamber is 66.8 μg/ml after 2 hours when it diffuses through a BC membrane of 3.3 mg/cm² at pH 2 and 4.6 μg/ml after 2 hours when it diffuses through a BC membrane of 13.9 mg/cm² at pH 2. Equilibrium of the diffusion is reached when the concentration of SRB in the receptor chamber is equal to the concentration of SRB in the donor chamber. The diffusion cell used in this study exhibits diffusion equilibrium around an SRB concentration of 1100 μg/ml. The concentrations 66.8 μg/ml and 4.6 μg/ml attained after 2 hours at pH 2 corresponds to around 6% (membrane 3.3 mg/cm²) and 0.5% (membrane 13.9 mg/cm²) of maximal SRB diffusion. 2 hours at pH 2 correspond to a typical maximal stomach passage time and a typical pH in the stomach. It can be concluded that bacterial cellulose can retain and protect an entrapped material during a stomach passage. The results in table 2 show that the BC membrane also retains and protect the entrapped material at pH 6.5 (corresponding to the pH in the intestine) and therefore requires a second component to speed up the release of the entrapped material. Diffusion through the membrane is dependent on the thickness of the membrane.

### Example 5

The purpose of this experiment was to determine the effect of pH on the activity of a cellulase complex introduced into a bacterial cellulose membrane.

Wet BC membrane was obtained from a culture of Komagataebacter xylinus and compressed to remove water. Celluclast 1.5 L (Novozymes, DK) cellulase complex solution from T. reesei (100 μl) was added to BC membranes and incubated to allow for the adsorption of the enzymes onto the BC fibers (15 min at 0° C.). The cellulase complex infused membranes were freeze-dried (3 days, −99° C.; 0.049 mbar). In preparations without infused cellulase the specific weight of the BC membrane was 3.1 mg/cm².

The digestion activity of the cellulase complex infused into BC membranes at pH 2 and pH 6.5 was investigated. The study was initiated by incubating the membranes at pH 2 and 6.5 (6.7 ml of 0.01 M HCl (pH 2) or PBS (pH 6.5) respectively). Samples were taken after 0 h, 1 h, 1.5 h, 2 h, 4 h and 6 h of cellulose digestion and analyzed for reducing sugars by the DNS method of Miller [13]. The results are shown in table 3.

TABLE 3
Reducing sugars released from a BC membrane by
cellulase complex digestion at pH 2 and pH 6.5

| | Cellulase complex | | Cellulase complex | |
|---|---|---|---|---|
| | activity at | | activity at | |
| | pH 2. | St. Dev. | pH 6.5 | St. Dev. |
| Digestion time | Reducing sugars | Reducing sugars | Reducing sugars | Reducing sugars |
| (h) | produced (mM) | produced (mM) | produced (mM) | produced (mM) |
| 0.0 | −0.03 | 0.01 | −0.03 | 0.01 |
| 1.0 | 0.49 | 0.05 | 0.96 | 0.04 |
| 1.5 | 0.51 | 0.04 | 1.42 | 0.06 |
| 2.0 | 0.53 | 0.04 | 1.80 | 0.03 |
| 4.0 | 0.53 | 0.08 | 2.29 | 0.13 |
| 6.0 | 0.55 | 0.04 | 2.40 | 0.13 |

The results show that a cellulase complex which is introduced into a cellulose membrane, freeze dried and then rehydrated can be brought to digest the cellulose membrane. This demonstrates the validity of the two-component coating production process shown in FIG. 2. At pH 2 corresponding to the environment in the stomach, the cellulase activity and therefore the cellulose digestion is inhibited and stops after 1 h. At pH 6.5, corresponding to the environment in the intestine, the BC membrane is digested.

### Example 6

The purpose of this experiment was to determine the effect of pH on the activity of a combination of cellulases introduced into a bacterial cellulose membrane.

Wet BC membrane was obtained from a culture of Komagataebacter xylinus and compressed to remove water. An 8:8:3:6 combination of cellulases was prepared from: 2 endo cellulases from Thermobifida fusca and Clostridium cellulolyticum (EC: 3.2.1.4 from family 6A and 9G respectively) and 2 exocellulases from Podospora anserina and Thermobifida fusca (EC: 3.2.1.91 family 6A and EC: 3.2.1.176 family 48A). The enzymes have activity optimum in the range pH 5.0 to 9.0, a cellulose binding domain and have crystalline cellulose as substrate. All enzymes were produced by heterologous expression followed by purification (NZYTech, Portugal).

The cellulase combination solution (415 μl) was added to compressed BC membranes and incubated to adsorb the enzymes to the BC fibers. (15 min at 0° C.). The BC membranes were freeze-dried (3 days, −99° C.; 0.049 mbar). In preparations without infused cellulase the specific weight of the BC membrane was 7.2 mg/cm².

The activity study was initiated by incubating the cellulase-combination infused freeze-dried BC membranes at pH 2 and 6.5 (6.7 ml of 0.01 M HCl (pH 2) or PBS (pH6.5) respectively). Samples were taken after 0 h, 1 h, 1.5 h, 2 h, 4 h and 6 h of digestion and the production of reducing sugars was measured by the method of Miller [13]. The results are shown in table 4.

TABLE 4
Digestion of BC membranes by a cellulase-combination at pH 2 and pH 6.5.

| | Cellulase | | Cellulase | |
|---|---|---|---|---|
| | combination | | combination | |
| | activity pH 2 | St. Dev. | activity pH 6.5 | St. Dev. |
| | Reducing sugars | Reducing sugars | Reducing sugars | Reducing sugars |
| Digestion time | produced | produced | produced | produced |
| (h) | (mM) | (mM) | (mM) | (mM) |
| 0 | −0.02 | 0.07 | 0.16 | 0.17 |
| 1.0 | −0.02 | 0.05 | 0.13 | 0.14 |
| 1.5 | 0.00 | 0.01 | 0.08 | 0.02 |
| 2.0 | −0.03 | 0.00 | 0.11 | 0.05 |
| 4.0 | −0.01 | 0.04 | 0.44 | 0.03 |
| 6.0 | −0.05 | 0.01 | 0.70 | 0.06 |

The results show that a cellulase combination which is introduced into a cellulose membrane, freeze dried and then rehydrated can be brought to digest the BC cellulose membrane. This further demonstrates the validity of the two-component coating production process shown in FIG. 2. At pH 2, corresponding to the environment in the stomach, the activity is strongly inhibited, and no activity can be detected. At pH 6.5, corresponding to the environment in the intestine, the BC membrane is digested.

### Example 7

The purpose of this experiment was to show the diffusion of a model compound through a BC membrane (component 1) digested by an introduced cellulase (component 2)

Wet BC membrane was obtained from a culture of Komagataebacter xylinus and compressed to remove water. Celluclast 1.5 L (Novozymes, DK) cellulase complex solution from T. reesei (100 μl) was added to BC membranes and incubated to allow for the adsorption of the enzymes onto the BC fibers (15 min at 0° C.). The cellulase complex infused BC membranes were freeze-dried (3 days, −99° C.; 0.049 mbar). In preparations without infused cellulase the specific weight of the BC membrane was 7.3 mg/cm². Sulforrodamin B (SRB) (MW 558.7 g/mol) was used as model compound. The diffusion over time of SRB through the BC membrans were investigated in a diffusion cell at pH 2 and pH 6.5. The donor chamber of the diffusion cell was filled with 2.9 mL of 2 mg/mL SRB solution at pH 2 or pH 6.5. The membrane to be studied was placed on top of the donor chamber. The study was initiated when the receptor chamber was filled with 0.01 M HCl (pH 2) or PBS (pH 6.5) as corresponding to the pH in the donor chamber. The diffusion cell was placed at 50° C. Samples were taken from the receptor chamber at 0 h, 1 h, 2 h, 3 h, 4 h and 6 h and the concentration of SRB was measured at OD 565 nm. The results are shown in table 5.

TABLE 5
Diffusion of a model compound through a BC membrane which
is, at the same time, digested by a cellulase complex
that was previously introduced into the membrane.

| | Concentration of SRB (μg/ml) in receptor chamber | | | |
|---|---|---|---|---|
| | Activity and | | Activity and | |
| Activity and | diffusion | St. Dev. | diffusion | St. Dev. |
| diffusion time | at pH 2 | SRB (μg/ml) | at pH 6.5 | SRB (μg/ml) |
| (h) | SRB (μg/ml) | pH 2 | SRB (μg/ml) | pH 6.5 |
| 0.0 | 0.2 | 0.0 | 0.0 | 0.0 |
| 1.0 | 0.4 | 0.1 | 22.5 | 3.1 |
| 2.0 | 0.9 | 0.2 | 62.7 | 4.0 |
| 3.0 | 6.2 | 3.9 | 118.3 | 8.1 |
| 4.0 | 12.7 | 6.7 | 211.3 | 35.6 |
| 6.0 | 30.1 | 10.7 | 588.0 | 201.6 |

The results show that diffusion of a model compound through a BC membrane digested by an infused cellulase complex is constant at a lower rate at pH 2 and is at a higher rate at pH 6.5. The results exemplify the use of a pair of components for preparing a coating where 1) the coating is digested, when subjected to the higher pH but not at the lower pH, 2) the first component (exemplified here by bacterial cellulose) is indigestible by the natural digestion in the gastro-intestinal system and 3) the second component (exemplified here by a cellulase) digest the first component when subjected to the higher pH in the intestine, but not when subjected to the lower pH in the stomach. Thus, the BC membrane coating prevents the release of the model compound under typical conditions found in the stomach (pH 2) and promotes the release of the model compound under typical conditions found in the intestine of a mammal (pH 6.5).

### CONCLUSION

Based on the studies presented the described coating material allows protection of the entrapped material in the stomach of a mammal, such as a human, and allows for release in the intestine. Together example 1 to 3 show that a high degree of robustness of design space can be obtained, allowing for a wide range of coating specifications. Together example 4 to 7 shows the technical feasibility of the described coating material.

### REFERENCES

[1] Dressman J B, Berardi R R, Dermentzoglou L C, Russell T L, Jarvenpaa K M. Upper Gastrointestinal (GI) pH in Young, Healthy Men and Women. Pharm Res. 1990; 7:756-761.
[2] Clarysse S, Tack J, Lammert F, Duchateau G, Reppas C, Augustijns P. Postprandial Evolution in Composition and Characteristics of Human Duodenal Fluids in Different Nutritional States. J. Pharm Sci. 2009; 98:1177-1192.
[3] Zarate N, Mohammed S D, O'Shaughnessy E, Newell M, Yazaki E, Williams N S, Lunniss P J, Semler J R, Scott S M. Am J Physiol—Gastroint and Liver Physiol 2010; 299 (6): G1276-G1286. Accurate localization of a fall in pH within the ileocecal region: Validation using a dual-scintigraphic technique.
[4] Worsøe et al. Gastric transit and small intestinal transit time and motility assessed by a magnet tracking system BMC Gastroenterology 2011, 11:145
[5] Assessment of regional gut transit times in healthy controls and patients with gastroparesis using wireless motility technology. I. Sarosiek et al. Aliment Pharmacol Ther. 2010 January 15; 31(2): 313-322
[6] Occurrence of cellulose, Kyle Ward, J R. In Cellulose and Cellulose Derivatives, Part I; Ott, E., Spurlin, H. M., Eds.; Interscience Publishers Inc. New York, 1954; Vol. V, p 9-29
[7] Ultrafine Cellulose Fibers Produced by Asaia bogorensis, an Acetic Acid Bacterium, A. Kumagai et al. Biomacromolecules 2011, 12, 2815-2821
[8] Sensing the structural differences in cellulose from apple and bacterial cell wall materials by Raman and FT-IR spectroscopy. Szymańska-Chargot M, Cybulska J, Zdunek A. Sensors (Basel). 2011; 11(6):5543-60.
[9] BRENDA, the enzyme information system in 2011. Nucleic Acids Res. 2011, 39: D670-D676, www.brenda-enzymes.org.
[10] A novel efficient β-glucanase from a paddy soil microbial metagenome with versatile activities. Biotechnol Biofuels. 2016 Feb. 13; 9:36
[11] Screening and characterization of a cellulase with endocellulase and exo-cellulase activity from yak rumen metagenome J. Mol. Catal. B 73, 104-110 (2011)
[12] Cloning and Characterization of an Endoglucanase Gene from Actinomyces sp. Korean Native Goat 40. Asian Australas. J. Anim Sci. Vol. 29, No. 1: 126-133 January 2016.
[13] Use of dinitrosalicylic acid reagent for determination of reducing sugar. Analytical Chemistry. 1959; 31(3):426-428.

1-21. (canceled)

22. A solid oral composition for targeted release in an intestine of a mammal comprising a core and a coating completely surrounding the core, wherein the core comprises an entrapped material to be released in the intestine, and wherein the coating or part of the coating comprises a first and a second component, wherein the first component is resistant to the environment in the stomach of a mammal and the second component enzymatically digest the first component when subjected to the more basic environment of the intestine compared to the more acidic environment of the stomach.

23. The composition of claim 22, wherein the coating is adapted to resist breakdown from the environment in a stomach of a mammal.

24. The composition of claim 22, wherein the coating prevents the release of the entrapped material in a stomach of a mammal.

25. The composition of claim 22, wherein the coating protects the entrapped material in a stomach of a mammal.

26. The composition of claim 22, wherein the coating is adapted to release the entrapped material in an intestine of a mammal.

27. The composition of claim 22, where the digesting activity of the second component is inhibited in the environment in the stomach of a mammal.

28. The composition of claim 22, wherein the mammal is selected from animals with little or no ability to digest cellulose, such as a human, a monkey, a pig, a dog, a human ape, a rodent and a cat.

29. The composition of claim 22, wherein the entrapped material is a medicinal product, such as a protein, an enzyme, a polypeptide, an oligopeptide, a peptide, a deoxyribonucleic acid (DNA), a ribonucleic acid (RNA), a small organic molecule of less than 900 Da, or a pro-drug of any one of these materials, is a nutritional supplement, or a microbial culture, or a microbial additive, or a vaccine.

30. The composition of claim 22, wherein the first component and the second component are mixed.

31. The composition of claim 22, wherein the first component and the second component are in different layers with the first component being the outer layer and the second component the inner layer around the core.

32. The composition of claim 22, wherein the pair of the first and second component is selected from cellulose and cellulase, pectin and pectinolytic enzymes, hemicellulose and hemicellulase, lignin and lignin degrading enzymes, fructan and fructan degrading enzymes, and lipid and lipases.

33. The composition of claim 22, wherein the pair of the first and second component is selected from structurally ordered cellulose I such as bacterial cellulose or derivatives thereof and a cellulase, such as cellulase (EC 3.2.1.4) (endocellulase), cellubiase (EC 3.2.1.21) (beta-glucosidase), 1,4-beta-cellobiosidase (EC 3.2.1.91) (Exocellulase), Cellulose 1,4-beta-cellobiosidase (reducing end) (EC 3.2.1.176) (exocellulase) as well as cellulase complexes and mixtures thereof.

34. The composition of claim 22, wherein the second component is enzymatically active and digests the first component in the environment in the intestine of a mammal.

35. The composition of claim 34, wherein the second component has maximum enzymatic activity in the range pH 3-12, such as pH 3.5 to 9.5, or 3.0 to 9.0, or 4.0 to 9.0.

36. The composition of claim 22, wherein the intestine is selected from small intestine, large intestine, duodenum, ileum, jejunum, and colon.

37. The composition of claim 22, wherein the composition is a tablet or a capsule or other type of solid oral dosage form.

38. The composition of claim 22 for use as a medicinal product.

39. The composition of claim 22 for use as a nutritional supplement.

40. A use of a pair of components for preparing a coating for a solid oral dosage form where the coating is degraded when subjected to a pH change from a lower to a higher pH, wherein the digestion of the first component by the second component is inhibited at the lower pH and the second component digest the first component when subjected to the higher pH.

41. The composition of claim 22, wherein the entrapped material is selected from:
a) Proteins, Human growth hormone (hGH), Calcitonin, Insulin, GLP-1 analogues, GLP-1
b) Peptides, Octreotide
c) Oral vaccines, Oral cholerae vaccine, Mycoplasma hyopneumoniae oral vaccine, Live bacterial cells as attenuated vaccines, live cell culture
d) Small organic molecules 200<MW<900 g/mol, Desmopressin, Vasopressin, Cyclosporine, Ranitidine, Diclofenac, Ketoprofen, Amifostine, Omeprazole, Gemcitabine, Domperidone, Paclitaxel, Cinnarizine, Donepezil, Leucovorin, Raloxifene, Indomethacin, Dextromethorphan, Nizatidine, Peptide Val-Leu-Pro-Valpro-Arg (VLPVPR), Flurbiprofen, Mebendazole, Thymidine, Zolpidem tartarate, Loratidine, Venlafaxine, Tamsulosin, Urapidil, Prednisolone, Miconazole, Diltiazem, Ambroxol, Captopril, Acyclovir, Cimetidine, Metoprolol, Griseofulvin, Atazanavir, Ibuprofen, Azithromycin, Lercanidipine, Sulfacetamide, Azelastine, Zidovudine, Cloricromene, Oxymatrine, Acarbose, Propranolol, Alfuzosin, Stavudine, Lobenzarit, Genistein, Verapamil, Terbinafine, Lornoxicam, Clotrimazole.
